(19) United States
(12) Patent Application Publication
(10) Pub. No.: US 2002/0193319 A1
(43) Pub. Date: Dec. 19, 2002

(54) Use of EGF genistein to prevent development of EGF-receptor expressing cancers

(75) Inventor: Fatih M. Uckun, White Bear Lake, MN (US)

Correspondence Address: MERCHANT & GOULD PC, P.O. BOX 2903, MINNEAPOLIS, MN, 55402-0903, US

(73) Assignee: Parker Hughes Institute, St. Paul, MN

(21) Appl. No.: 10/145,798

(22) Filed: May 14, 2002

Related U.S. Application Data
(63) Continuation of application No. PCT/US00/31299, filed on Nov. 15, 2000.
(60) Provisional application No. 60/165,544, filed on Nov. 15, 1999.

(51) Int. Cl. A61K 31/7048; A61K 31/353

(52) U.S. Cl. 514/27; 514/456

(57) ABSTRACT

The invention provides a method for preventing the development or recurrence of cancer in a mammal by administering to the mammal a compound comprising an isoflavone, such as Genistein, conjugated to epidermal growth factor (EGF).

This application is being filed as a PCT International Patent Application in the name of Parker Hughes Institute, a U.S. national corporation, (Applicant for all countries except US), and Fatih M. Uckun, a U.S. citizen (Applicant for US only), on Nov. 15, 2000, designating all countries.

### BACKGROUND OF THE INVENTION

Human epidermal growth factor (EGF) is a six kilodalton (kDa) 53 amino acid, single-chain polypeptide that exerts its biological effect by binding to a specific 170 kDa cell membrane receptor, EGF-receptor/Erb-1 (EGF-Rc). Human EGF-Rc includes an extracellular domain with high cysteine content and N-linked glycosylation, a single transmembrane domain, and a cytoplasmic domain with protein tyrosine kinase (PTK) activity. Binding of EGF to the EGF-Rc results in receptor dimerization with itself or other members of the ErbB (subtype I) transmembrane PTK family (including Erb-B2 and Erb-B3), resulting in activation and autophosphorylation of the PTK domain. EGF-Rc is physically and functionally associated with the Src protooncogene family PTK including p60 Src. This association is believed to be an integral part of the signaling events mediated by the EGF-Rc.

Many types of cancer display enhanced EGF-Rc expression on their cell surface membranes. It is believed that enhanced expression of EGF-Rc increases signaling via receptor-mediator pathways that lead to excessive proliferation and metastasis. Examples of cancers displaying enhanced EGF-Rc expression include prostate cancer, breast cancer, lung cancer, head and neck cancer, bladder cancer, melanoma, and brain tumors. Src kinase is believed to play a role in the pathogenesis of breast cancer, as the enzymatic activity of Src in breast cancer is significantly higher when compared to benign or normal breast tissue. Additionally, in breast cancer, expression of the EGF-Rc is a significant and independent indicator for recurrence and poor relapse-free survival.

Genistein, an isoflavone (5,7,4′-trihydroxyisoflavone), is a naturally occurring tyrosine kinase inhibitor present in soybeans, soy meal, and tofu. Genistein has been shown to prevent apoptosis in cells that have undergone ionizing radiation or engagement of the CD 19 receptor (Uckun et al., 1992, PNAS USA 89:9005). Genistein has also been shown to inhibit the in vitro proliferation of cancer cells, including human breast cancer cells (Monti et al., 1994, AntiCancer Res. 14:1221-1226).

Conjugates formed with isoflavaones such as Genistein and EGF have been shown to inhibit the EGF receptor tyrosine kinase in breast cancer cells, leading to apoptosis and cell death. See, U.S. Pat. No. 5,911,995.

### SUMMARY OF THE INVENTION

It has now been discovered that EGF-Isoflavone conjugates are effective agents for preventing the development or recurrence of cancer in a mammal, particularly cancer expressing EGF-Rc. In the method of the invention, a compound comprising an isoflavone, such as Genistein, conjugated to epidermal growth factor (EGF), when administered to a patient, effectively prevents the development or recurrence of cancer. In particular, the invention provides a method for preventing the development or recurrence of cancer in a mammal by administering to the mammal an effective cancer-preventing amount of a compound that inhibits the epidermal growth factor receptor (EGF-Rc) tyrosine kinase or a Src family kinase. More particularly, the invention provides a method to prevent the development or recurrence of an EGF-Rc expressing breast cancer in a mammal by administering to the mammal an effective cancer-preventing amount of a compound that inhibits the EGF-receptor tyrosine kinase, for example, EGF-Genistein, or a pharmaceutically acceptable salt thereof.

### BRIEF DESCRIPTION OF THE FIGURES

FIG. 1 is a photograph of a gel showing the intranucleosomal DNA fragmentation of cells treated with EGF-Genistein;

FIG. 2 is a graph showing clonogenic cell survival curves of human breast cancer cells treated with EGF-Genistein;

FIGS. 3A and 3B are bar graphs showing the in vivo effect of EGF-Genistein on established tumor xenografts in SCID mice;

FIG. 4 is a graph showing prevention of tumor growth in SCID mice inoculated with breast cancer cells and administered EGF-Genistein;

FIG. 5 shows an RNA blot analysis of RNA isolated from the mammary glands of 5 different Neu transgenic mice;

FIG. 6 is a photograph of a gel showing the expression of EGF-Rc in mammary glands and mammary tumors; and

FIG. 7 is a graph showing prevention of tumor growth in Neu/erbB2 transgenic mice administered EGF-Genistein.

### DETAILED DESCRIPTION

The invention provides a method for preventing the development or recurrence of cancer in a mammal. More specifically, the invention includes administering a compound comprising an isoflavone, such as Genistein, conjugated to epidermal growth factor (EGF) to a patient, or a pharmaceutically acceptable salt thereof.

Protein Tyrosine Kinases

Cell growth is controlled, to a large degree, by extracellular ligands that bind to specific receptors on the surface of cells. A number of these receptors, including the EGF receptor (EGF-Rc), have intrinsic protein tyrosine kinase (PTK) activity.

Protein tyrosine kinases (PTKs) appear to play key roles in the initiation of various signaling cascades. PTKs can be divided into two major groups. The first group includes receptor PTKs, such as EGF-Rc. The second group includes non-receptor PTKs, including the Src family of PTKs. Generally, the non-receptor PTK is associated with some type of cell surface ligand-binding protein, for example, EGF-Rc.

The Src family of non-receptor PTKs includes Src, Yes, Fyn, Lyn, Lck, Hck, Fgr, Blk, and Yrk. Src is expressed by cells associated with colon cancer, breast cancer and ovarian cancer, as well as cells associated with other forms of human cancer.

Isoflavone Protein Tyrosine Kinase Inhibitors

Several isoflavone compounds have been reported to inhibit tyrosine kinase activity. As used herein, the term “isoflavones” includes, but is not limited to, compounds found in a variety of leguminous plants, including soybeans, such as Genistein, Genistin, 6-acetate ester Genistin, Daidzein, Daidzin, 6-acetate ester Daidzin, Biochanin A, Glycitin, Formononetin and Coumestrol. Genistein (5,7,4′-trihydroxyisoflavone), Daidzein (7,4′-dihydroxyisoflavone), and Biochanin A (4-methoxygenistein) have been shown to inhibit proliferative growth of human breast cancer cell lines (Peterson, et al., 1991, BBRC 179:661-667).

Preferred compounds for use in the method invention are isoflavones, for example, having the general structure shown in I, below:

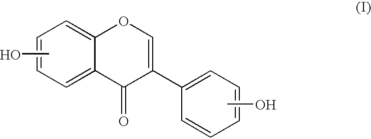

A preferred isoflavone is genistein, having the structure shown in II, below:

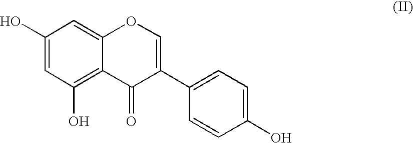

Genistein

Genistein (Gen) (5,7,4′-trihydroxyisoflavone) is a naturally occurring tyrosine kinase inhibitor present in soybeans (Uckun et al., 1995, Science 267:886-891). Genistein may be obtained commercially from Calbiochem (LaJolla, Calif.). Alternately, Genistein may be isolated from soybeans, soy meal, or tofu by the method described in Akiyma et al., 1987, J Bio Chem 272:5592. Genistein may also be synthesized as described in U.S. Pat. No. 5,911,995.

Epidernal Growth Factor (EGF)

Human epidermal growth factor (EGF) is a six kilodalton (kDa), 53 amino acid, single-chain polypeptide that exerts its biological effect by binding to a cell membrane receptor, EGF-receptor (EGF-Rc). Epidermal growth factor (EGF) is commercially available in a highly purified form, for example, from Upstate Biotechnology, Inc. (Lake Placid, N.Y.).

Epidermal Growth Factor Receptor (EGF-Rc)

EGF-receptor (EGF-Rc) is a 170 kDa cell membrane receptor that plays a role in human cancer. Generally, expression of EGF-Rc is increased in EGF-responsive cells. In cancer cells, the EGF-Rc associates with specific tyrosine kinases, including members of the Src protooncogene tyrosine kinase family. These membrane-associated complexes are vital regulators of cancer cell survival and prevention of programmed cell death (“apoptosis”). A more detailed discussion of the EGF-Rc is found in U.S. Pat. No. 5,911,995.

EGF-Rc is over-expressed in cancer cells, for example, in tumors of the brain, bladder, breast, stomach, cervix, and ovary. In breast, lung, and bladder tumors, over-expression of the EGF-Rc is an indicator of poor prognosis. It has now been found that inhibition of epidermal growth factor receptor (EGF-Rc) tyrosine kinase or of a Src family tyrosine kinase, for example, by administering an EGF-isoflavone such as EGF-Genistein, effectively prevents the development or recurrence of an EGF-Rc expressing cancer.

EGF-Isoflavone Conjugate

Methods for preparing EGF-Isoflavone conjugates are known. For example, EGF-Genistein can be formed by linking Genistein to a molecule of EGF as described in U.S. Pat. Nos. 5,911,995 and 6,034,053. A photochemical conjugation method, useful for producing EGF-Genistein, is described by Uckun et al., 1995, Science 267:886. Other isoflavones can be conjugated to EGF using similar methods.

EGF-Isoflavone conjugates have been shown to inhibit EGF-receptor tyrosine kinase and associated Src family tyrosine kinases, leading to apoptosis in human breast cancer cell both in vitro and in vivo (Uckun et al., 1998, Clinical Cancer Research, 4(5):1125-1134; and Uckun et al., 1998, Clinical Cancer Res. 4:901-912).

The inventors have now found that EGF-Isoflavone conjugates, EGF-Genistein for example, are also effective in preventing the development or recurrence of cancer cells that express EGF-Rc. EGF-Isoflavone conjugate is believed to specifically bind to EGF receptors (EGF-Rc) to inhibit EGF-Rc kinase and associated Src family tyrosine kinases.

Cancer

Carcinogenesis is a multistep process at both the phenotypic and genetic level. A malignant neoplasm has several phenotypic characteristics, such as excessive growth, local invasiveness and metastasis. These characteristics are acquired in a stepwise fashion, called tumor progression.

“Neoplasia” literally means “new growth.” A neoplasm, as used herein, refers to an abnormal mass of tissue, the growth of which exceeds and is uncoordinated with that of the normal tissues and persists in the same excessive manner after the cessation of the stimuli that evoked the change. Fundamental to the growth of neoplasms is the loss of responsiveness to normal growth controls. Neoplastic cells are “transformed” because they continue to replicate, apparently unaffected by the regulatory influences that control normal cell growth.

A neoplasm can be referred to as a “tumor.” A tumor is “benign” when its microscopic and gross characteristics are relatively innocent, e.g., it will likely remain localized and is amenable to local surgical removal. Patient survival is likely.

The term “cancer” refers to a malignant tumor. Malignant means that the neoplasm can invade and destroy adjacent structures and spread to distant sites (metastasize). Malignant tumors may cause death.

The determination of the nature of a tumor (e.g., benign or malignant) is predicted based on clinical and anatomic criteria. Generally, benign neoplasms are composed of differentiated cells that resemble their normal counterparts. Malignant neoplasms are characterized by a wide range of differentiation, from well-differentiated to completely undifferentiated. However, malignant tumors always display some loss of differentiation. Generally, benign tumors grow more slowly than malignant tumors. Malignant tumors eventually metastasize. The rate of growth of malignant tumors tends to correlate with their level of differentiation. A benign neoplasm remains localized at its site of origin and does not have the capacity to infiltrate, invade or metastasize to distant sites. Cancers grow by progressive infiltration, invasion, destruction, and penetration of the surrounding tissue.

Transformation is generally due to non-lethal genetic cellular damage. Genetic damage (mutations) may be acquired by the action of environmental agents, such as chemicals, radiation, or viruses, or may be inherited. Generally, a tumor mass results from the clonal expansion of a single progenitor cell that has incurred genetic damage (e.g., tumors are generally monoclonal). Three classes of normal regulatory genes, the growth promoting oncogenes, the growth-inhibiting cancer suppressor genes, and genes that regulate programmed cell death (apoptosis), are the principal targets of genetic damage.

Oncogenic transformation typically alters the pattern of expression of selected Src family members. Furthermore, tyrosine-specific protein kinase activity is generally associated with oncogene products of the Src gene family.

### Method of the Invention

The invention provides a method for preventing the development or recurrence of cancer in a mammal.

The invention includes administering a compound comprising an isoflavone, such as Genistein, conjugated to epidermal growth factor (EGF) to a patient to prevent the development or recurrence of cancer. In particular, the invention provides a method for preventing the development or recurrence of cancer in a mammal by administering to the mammal an effective cancer-preventing amount of a compound that inhibits the epidermal growth factor receptor (EGF-Rc) tyrosine kinase or a Src family tyrosine kinase. More particularly, the invention provides a method to prevent the development or recurrence of EGF-Rc expressing cancer such as breast cancer in a mammal by administering to the mammal an effective cancer-preventing amount of a compound that inhibits the EGF-receptor tyrosine kinase, for example, EGF-Genistein, or a pharmaceutically acceptable salt thereof.

The EGF-Genistein conjugate can be administered to treat premalignant conditions and to prevent progression to a neoplastic or malignant state. Such prophylactic or therapeutic use is indicated in conditions known or suspected for progression to neoplasia or cancer.

As used herein, the term “premalignant” condition refers to a condition that may or is likely to become cancer. Generally, a premalignant condition refers to a condition wherein a cell contains a genomic aberration that is likely to develop into cancer. For example, over-expression of EGF-Rc is correlated with development of specific cancers, and can be considered a premalignant condition. A premalignant condition can also refer to a benign tumor containing transformed cells that undergo rapid growth, or even a tumor that has invaded local tissue, but not yet metastasized.

As used herein, the term “preventing” generally refers to a process that reduces the likelihood or probability of an event. For example, as used herein, the phrase “preventing the development” of cancer refers to a process that slows or stops tumor progression. As discussed above, carcinogenesis is a multistep process, starting with cellular transformation and excessive growth, and leading to local invasivenss, metastasis, and even patient death. Thus, “preventing the development” of cancer refers to a process that prevents the tumor from progressing to the next step. Generally, when the phrase “preventing the development” of cancer is used herein, it is referring to a process that reduces the likelihood of a tumor progressing to metastasis and/or patient death.

As used herein, the phrase “preventing the recurrence” of cancer refers to a process that reduces the likelihood of cancer re-growth after treatment, for example, after the cancerous tumor has been surgically removed or destroyed (for example, by chemotherapy, radiation, etc.). One embodiment of the invention is the administration of an EGF-Isoflavone conjugate to prevent metastasis of a tumor and/or to prevent regrowth of cancer after surgical or chemical debulking or other treatment. The EGF-Isoflavone conjugate can be administered concurrently with such cancer treatment, or post-treatment.

“Therapeutically effective” refers to the inhibition, to some extent, of growth of cells causing or contributing to cancer. A therapeutic effect relieves, to some extent, one or more of the symptoms of cancer, such as a reduction of the number of cells, reduction in tumor size, or reduction of metastasis.

Administration of EGF-Isoflavone Conjugate

EGF-Isoflavone conjugate can be formulated as a pharmaceutical composition. Suitable administration routes include oral, intravenous, intramuscular, intraperitoneal, subcutaneous, or local delivery via an implantable device.

Suitable pharmaceutical dosage forms include formulations suitable for injection or infusion. Such formulations include sterile, aqueous solutions or dispersions or sterile powders. The carrier or vehicle can be a solvent or liquid dispersion medium comprising, for example, water, ethanol, a polyol such as glycerol, propylene glycol, or liquid polyethylene glycols, vegetable oils, nontoxic glyceryl esters and suitable mixtures thereof The fluidity can be maintained, for example, by the formation of liposomes, by the maintenance of particle size (in the case of a dispersion), or by the use of nontoxic surfactants. Various antibacterial and antifungal agents, for example, parabens, chlorobutanol, sorbic acid, and thimeosal, can prevent microbial growth. It may also be desirable to include isotonic agents in the formulation, for examples, sugars, buffers or sodium chloride. Agents that delay absorption may also be included, for example, aluminum monostearate hydrogels or gelatin. Pharmaceutical formulations for EGF-Genistein are discussed in U.S. Pat. No. 5,911,995.

The appropriate dosage can vary widely depending on the size, age and condition of the patient being treated. Useful dosages are those that yield a systemic exposure level (i.e., area under serum concentration×time curve) of 0.28 μg /L×hr or greater. Systemic exposure levels can be optimized in an individual patient by simply adjusting the dose according to the measured conjugate concentration in the serum.

The EGF-Isoflavone conjugates can be administered alone or in combination with other cancer prevention therapies. For example EGF-Genistein can be administered in combination with other compounds that inhibit tyrosine kinase activity or with other chemotherapeutic agents such as doxorubicin, cyclophosphamide, methotrexate, 5-fluorouracil, mitomycin C, mitoxantrone, taxol, and epirubicin.

### EXAMPLES

The invention will be further described by reference to the following detailed examples. Although the examples use one specific isoflavone, Genistein, it is expected that additional isoflavones can be similarly made and used.

### Example 1

### Preparation of EGF-Genistein

EGF-Genistein was prepared as described in Example 1 of U.S. Pat. No. 5,911,995.

### Example 2

### EGF-Genistein Induction of Apoptosis in Human Breast Cancer Cells.

Apoptosis

Apoptosis, or programmed cellular death, culminates in the activation of endogenous endonucleases that degrade the DNA of the cell, thereby destroying the genetic template required for cellular homeostatis. Apoptosis is observed in controlled deletion of cells during metamorphosis, differentiation, and general cell turnover and appears normally to be regulated by receptor coupled events.

Apoptotic cell death is characterized by plasma membrane blebbing, cell volume loss, nuclear condensation, and endonucleolytic degradation of DNA. Loss of plasma membrane integrity is a relatively late event in apoptosis, unlike the form of cell death termed necrosis, which can be caused by hypoxia and exposure to certain toxins and which is typically characterized early on by increased membrane permeability and cell rupture.

Fluorescence Activated Cell Sorting (FACS)

Fluorescence activated cell sorting (FACS) was used to sort MDA-MB-231 cells (breast cancer cells expressing EGF-Rc). The cells were treated with either PBS EFG-Genistein (0.1, 1, and 10 μM) or unconjugated Geninstein (1, 10, and 100 μM) and incubated for 24 hours. The cells were then stained with MC-540 and propidium iodide. Single MC540 fluorescence (S-MC540) indicates the cell is at an early stage of apoptosis. Dual MC540/propidium iodide fluorescence (D-MC540/PI) indicates the cell is at an advanced stage of apoptosis. The total apoptotic fraction (TAF) is calculated as the percentage of MC540 fluorescent cells plus the percentage of MC540/propidium iodide double fluorescent cells.

The results, shown in Table 1 below, demonstrate the superior efficacy of EGF-Genistein in inducing apoptosis in cancer cells as compared to unconjugated EGF.

TABLE 1
Effect of EGF-Genistein on apoptosis in human breast cancer cells

| | Control | 0.1 μM | 1 μM | 10 μM | 100 μM |
|---|---|---|---|---|---|
| EGF | | | | | |
| (% TAF) | 0.8 | — | 6.3 | 10.7 | 15.5 |
| (% S-MC540) | 0.7 | — | 5.5 | 9.5 | 13.5 |
| (% D-MC540/PI) | 0.1 | — | 0.8 | 1.2 | 2.0 |
| EGF-Genistein | | | | | |
| (% TAF) | 2.2 | 98.7 | 97.6 | 98.9 | — |
| (% S-MC540) | 1.4 | 78.8 | 61.0 | 42.2 | — |
| (% D-MC540/PI) | 0.8 | 20.0 | 36.6 | 56.7 | — |

*% TAF: Single MC540 plus Dual MC540/propidium iodide
% S-MC540: Single MC540
% D-MC540/PI: Dual MC540/propidium iodide

Intranucelosomal DNA Fragmentation

As discussed above, apoptosis culminates in the degradation of cellular DNA. Therefore, intranucleosomal DNA fragmentation was used to monitor the apoptotic response to EGF-Genistein. Intranucleosomal DNA fragmentation assays are known. See, for example, Waddick et al., 1995, Blood 86:4228-4233.

Briefly, MDA-MB-231 and BT-20 cells (both cell types are breast cancer cell lines that express EGF-Rc) were cultured for 24 hours in the presence of PBS (control); Genistein conjugated to Granulocyte Colony Stimulating Factor (G-CSF-Gen) (10 μg/ml); unconjugated EGF (10 μg/ml) plus unconjugated Genistein (10 μg/ml); conjugated EGF-Genistein (1 μg/ml); or conjugated EGF-Genistein (10 μg/ml). Cellular DNA was prepared for analysis of fragmentation and separated by electrophoresis using a 1% agarose gel. The DNA bands were stained with ethidium bromide and visualized using UV light.

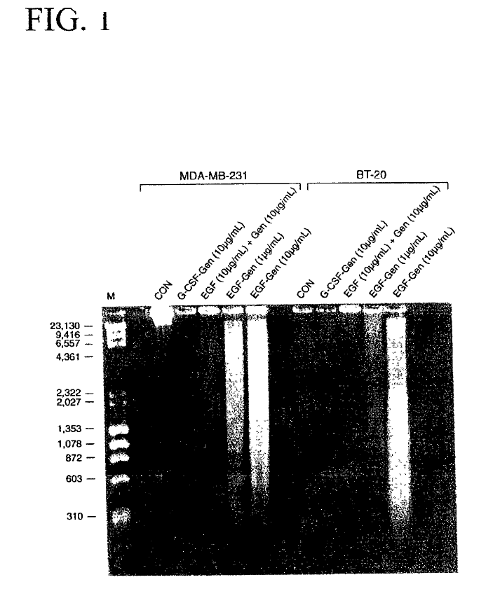

The results, shown in FIG. 1, demonstrate that cells treated with the EGF-Genistein conjugate displayed intranucleosomal DNA fragmentation, indicating that EGF-Genistein induced apoptosis in human breast cancer cells. Control and G-CSF-Genistein did not induce apoptosis. The molecular size markers (in bp) are shown in lane M.

### Example 3

### EGF-Genistein Cytotoxicity Against Clonogenic MDA-MB-231 and BT-20 Human Breast Cancer Cells

MDA-MB-231 and BT-20 human breast cancer cells were treated with 0.1, 0.3, 1,3, 10, 30, or 100 μM EGF-Genistein or equimolar concentrations of unconjugated Genistein for 24 hours. Cells were then assayed for clonogenic growth in vitro. Composite cell survival curves were generated using the dose-response data from three independent experiments, each performed in duplicate.

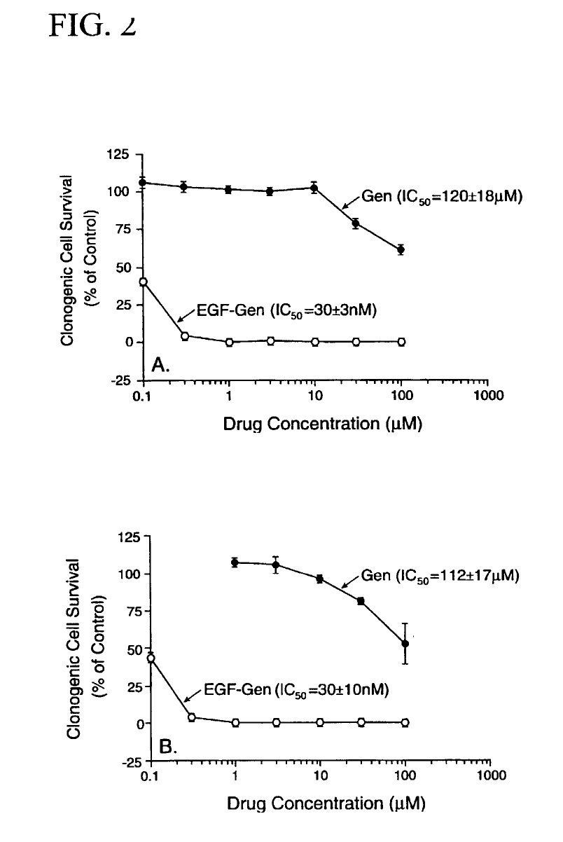

The results are shown in FIG. 2. Panel A shows the results for MDA-MB-231 cells. Panel B shows the results for BT-20 cells. The clonogenic cell survival for both cell types was significantly less for the cells treated with EGF-Genistein than for the cells treated with unconjugated Genistein.

### Example 4

### In vivo Activity of EGF-Gen Against Established Tumors

The effect of EGF-Genistein conjugate on the growth of established tumors was examined using a SCID mouse xenograph model. EGF-Genistein was not toxic to SCID mice.

SCID mice were inoculated subcutaneously with human breast cancer (MDA-MB-231) xenografts having a diameter of 0.5 cm or 1.0 cm, and then treated with 100 μg/kg/day EGF-Genistein intraperitoneally for 10 consecutive days. The tumor diameter was determined daily for 20 days from the start of therapy. Control mice were treated with 0.2 ml of PBS for 10 consecutive days or unconjugated Genistein at a concentration of 500 μg/kg/day for 10 days. P values were determined using Student's t test.

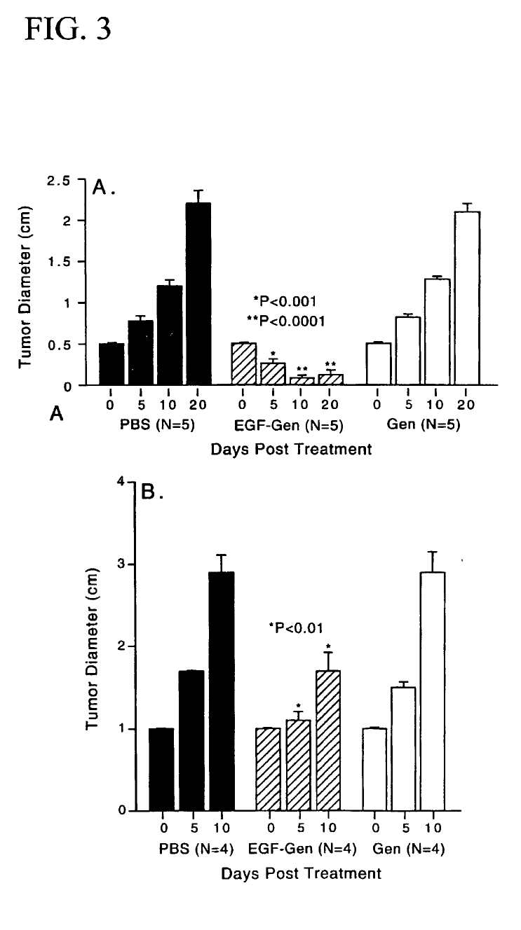

The results in FIG. 3A (0.5 cm xenograft) and 3B (1.0 cm xenograft) show that mice treated with EGF-Genistein exhibited a reduction in tumor diameter as compared to mice treated with PBS or unconjugated Genistein. Thus, administration of EGF-Genistein slowed, and in the case of the smaller 0.5 cm xenograft, reversed, tumor progression.

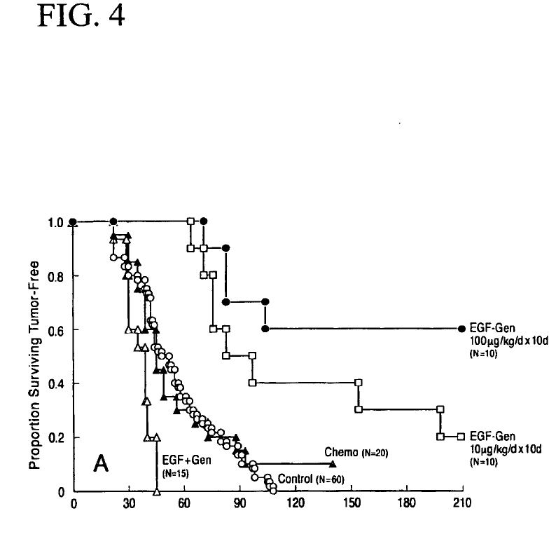

In another experiment, SCID mice were inoculated subcutaneously with 1×10⁶ MDA-MB-231 cells. Twenty-four hours after inoculation, the mice received conjugated EGF-Genistein (10 μg/kg/day for 10 days, n=10, or 100 μg/kg/day for 10 days, n=10); unconjugated EGF (500 μg /kg/day for 10 days) plus unconjugated Genistein (500 μg/kg/day for 10 days, n=15), or chemotherapy (n=20, i.e., cyclophosphamide, 50 mg/kg/day for 2 days; adriamycin, 2.5 mg/kg/day single bolus dose; or methotrexate, 0.5 mg/kg single bolus dose). Controls (n=60) were treated with PBS, G-CSF-Genistein (500 μg/kg/day for 10 days) or Genistein (500 μg/kg/day for 10 days). Tumor-free survival curves are shown in FIG. 4. Tumor growth was reduced in the mice receiving EGF-Genistein. These mice had a significantly higher tumor free-survival rate than the other mice, both initially and at 210 days.

### Example 5

### Expression of unactivated Neu Transgene in the Mammary Gland of Neu Transgenic Mice

RNA Blot Analysis

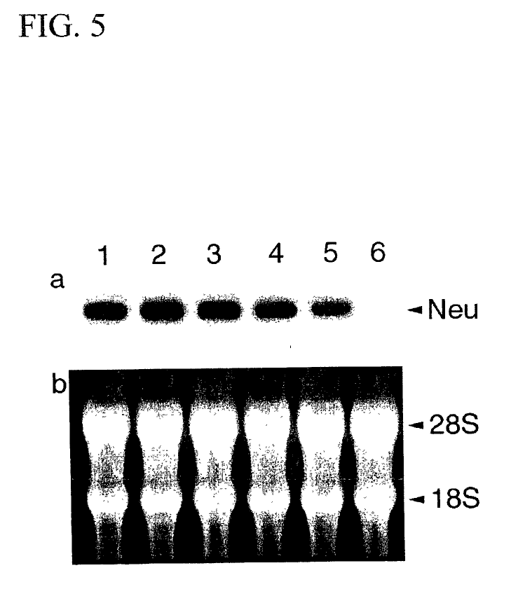

25 mg of total RNA was isolated from the mammary gland of 5 different Neu transgenic mice at day 2 of lactation and analyzed by RNA blot analysis. The RNA was probed with Neu and stained with ethidium bromide to demonstrate equivalent loading of total RNA. The results are shown in FIG. 5. Panel a was probed with Neu and was exposed for 1.5 hours. Panel b is the ethidium bromide stained RNA gel. Lanes 1-5 were 5 total RNA samples isolated from 5 different Neu bitransgenic mice. Lane 6 was a control containing total RNA from a non-transgenic mouse. All five Neu transgenic mice expressed the Neu transgene.

### Example 6

### Expression of EGF-Rc in Mammary Glands and Mammary Tumors of Neu Mice

RT-PCR

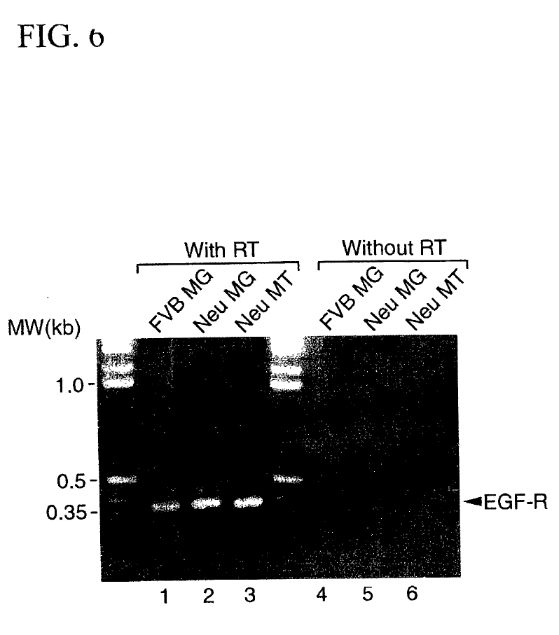

The expression of EGF-Rc in mammary glands and mammary tumors was examined by RT-PCR (reverse transcriptase polymerase chain reaction). Total RNA was isolated from mammary glands (FVB transgenic mice and Neu mice) and mammary tumor (Neu mice) and subjected to RT-PCR. The results are shown in FIG. 6. Lane 1, 2 and 3 are the RT-PCR results of RNA from FVB mammary gland, Neu mammary gland and Neu mammary tumor, respectively. All three express EGF-Rc. Control lanes 4, 5 and 6 show the PCR results on the same RNA samples using a RT-free reaction mixture.

Immunohistochemical Staining

Expression of EGF-R in the lactating mammary glands and mammary tumor in FVB non-transgenic mice and Neu transgenic mice was examined by immunohistochemical staining. (Results not shown) The staining showed high levels of EGF-Rc expression in the epithelial cells of lactating mammary glands of non-transgenic and Neu transgenic mice. EGF-Rc expression was also very strong in the mammary tumor cells of Neu transgenic mice.

### Example 7

### EGF-Genistein Prevented Spontaneous Development of Breast Cancer in Neu/erbB2 Transgenic Mice.

The ability of EGF-Genistein to prevent spontaneous development of breast cancer in neu/erbB2 transgenic mice was examined. Neu/erbB2 transgenic mice were chosen because they overexpress the Neu protooncogene in their mammary glands.

Starting when the transgenic mice were 4 months old, 10 Neu/erbB2 mice were administered 100 μg EGF-Genistein subcutaneously 1×per week for 10 weeks (dose level was 5 mg/kg). As a control, 29 Neu/erbB2 mice were treated with PBS.

All 29 PBS-treated control mice developed breast cancer with a median cancer free survival of 210 days. In contrast, only 40% of EGF-Genistein treated mice developed breast cancer and the median cancer-free survival was >300 days. EGF-Genistein treatment was not associated with any side effects.

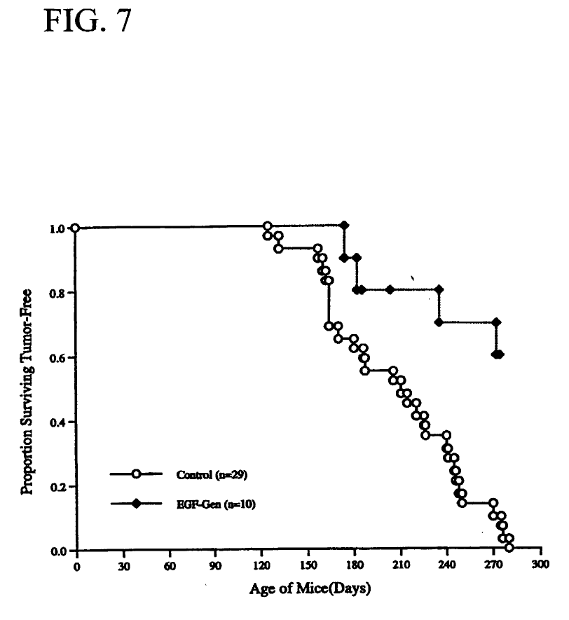

Tumor-free survival curves of new transgenic mice were generated and are shown in FIG. 7. The data was expressed as the proportion of mice surviving tumor free.

This data indicates that EGF-Genistein delayed onset of spontaneous growth of Neu mediated mammary tumors in Neu transgenic mice. EGF-Genistein may be useful for chemoprevention of EGF-receptor expressing breast cancer and/or its recurrence.

Table 2, below, shows Life table analysis chemopreventive activity of EGF-Gen in a neu transgenic mouse model of breast cancer.

TABLE 2
Life table analysis chemopreventive activity of EGF-Gen in a neu
transgenic mouse model of breast cancer

| | Proportion Surviving | |
|---|---|---|
| | Control (n = 29) | EGF-gen (n = 10) |
| 120 days | 100% | 100% |
| 150 days | 93.1 ± 4.7% | 100% |
| 180 days | 62.1 ± 9.0% | 90 ± 9.5% |
| 210 days | 48.3 ± 9.3% | 80 ± 12.6% |
| 240 days | 31.0 ± 8.6% | 53.3 ± 23.3% |
| 270 days | 10.3 ± 5.7% | 53.3 ± 23.3% |

* The 4-month-old neu transgenic mice were injected i.p. Once a week with EGF-Gen (100 μg/week × 10 weeks; n = 10) or control mice treated with PBS (n = 29) and the health status was monitored.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for preventing development of EGF-receptor expressing cancer in a subject, comprising administering to the subject an effective preventative amount of an EGF-isoflavone.

2. A method for preventing reoccurrence of EGF-receptor expressing cancer in a subject, comprising administering to the subject an effective preventative amount of EGF-isoflavone.

3. A method for preventing metastasis of EGF-Rc expressing cancer in a subject, comprising administering to the subject an effective preventative amount of EGF-isoflavone.

4. A method for inhibiting Src family tyrosine kinase in an EGF-Rc expressing cancer cell, comprising administering to the cell an effective inhibitory amount of an EGF-isoflavone.

5. The method according to claim 1, 2, 3 or 4, wherein EGF-isoflavone is selected from the group consisting of EGF-Genistein, EGF-Daizden, EGF-Biochanin and EGF-aminogen.

6. The method according to claim 1, 2, 3 or 4, wherein EGF-isoflavone comprises EGF-Genistein.

7. The method according to claim 1, 2, 3 or 4, wherein the cancer is breast cancer.

8. The method according to claim 1, 2, 3 or 4, wherein the step of administering is conducted concurrent with debulking treatment.

9. The method according to claim 1, 2, 3 or 4, wherein the step of administering is conducted after debulking treatment.

10. A method of preventing development or recurrence of cancer in a mammal, the method comprising:
administering to the mammal a therapeutically effective amount of a compound comprising EGF-Genistein.
